(19) United States
(12) Patent Application Publication
(10) Pub. No.: US 2025/0341515 A1
(43) Pub. Date: Nov. 6, 2025

(54) METABOLOMIC BIOMARKERS FOR ACUTE KIDNEY INJURY, ASSAYS, COMPOSITIONS AND METHODS OF USE THEREOF

(71) Applicant: Wisconsin Alumni Research Foundation, Madison, WI (US)

(72) Inventors: Joshua Coon, Middleton, WI (US); Matthew Harer, Waunakee, WI (US); Nicole Nightingale, Saint Paul, MN (US); Chase Robert Schoenenberger, Madison, WI (US); Paige E Condit, Madison, WI (US); Katherine Overmyer, Madison, WI (US)

(73) Assignee: Wisconsin Alumni Research Foundation, Madison, WI (US)

(21) Appl. No.: 19/194,599

(22) Filed: Apr. 30, 2025

Related U.S. Application Data
(60) Provisional application No. 63/641,055, filed on May 1, 2024.

(51) Int. Cl. G01N 33/53

(52) U.S. Cl. CPC G01N 33/5308; G01N 2800/347; G01N 2800/52

(57) ABSTRACT

Described herein is a method of determining a subject has AKI including providing a urine sample from the subject, and detecting an AKI metabolomic biomarker in the urine sample. Also described herein is a method of evaluating the effect of AKI therapy in a subject with AKI including administering the AKI therapy to the subject, providing a urine sample from the subject after the administering, and detecting an AKI metabolomic biomarker in the urine sample. Methods of method of identifying metabolomic biomarkers associated with acute kidney injury (AKI) in preterm neonates are also described.

### CROSS-REFERENCE TO RELATED APPLICATIONS

This application claims priority to U.S. Provisional Application 63/641,055 filed on May 1, 2024, which is incorporated herein by reference in its entirety.

### STATEMENT REGARDING FEDERALLY SPONSORED RESEARCH & DEVELOPMENT

This invention was made with government support under GM108538 and GM118110 awarded by the National Institutes of Health. The government has certain rights in the invention.

### FIELD OF THE DISCLOSURE

The present disclosure is related to the identification of metabolomic biomarkers for acute kidney injury, particularly in infants born prematurely, and assays, methods, and compositions for detecting the metabolomic biomarkers.

### BACKGROUND

The filtering and functional unit of the kidney is the nephron. Nephrons continue to form until 36 weeks gestation, mature until 2 years after birth, and no new nephrons are formed after that time. Thus, in premature birth, the development of nephrons is truncated and premature infants start life with fewer nephrons than term infants. According to Brenner's hypothesis, this congenital reduction in nephron number puts premature infants at higher risk for chronic kidney disease (CKD). Several childhood studies have confirmed this hypothesis and found higher rates of hypertension, proteinuria and reduced estimated glomerular filtration rates in former premature infants. Acute kidney injury (AKI) is particularly common in premature infants in the NICU, occurring in up to 50% of infants born <29 weeks gestation. Those with AKI have over 4 times the risk of death in the NICU and have at least 8 days longer hospitalizations. AKI was previously thought to be a reversible phenomenon with no long-term effects on renal function. However, over the past decade, multiple adult, pediatric and neonatal studies have demonstrated that AKI is not reversible, and that there are permanent decreases in renal function that result in CKD during childhood.

Given the long-term impact of neonatal AKI, a group of experts met in 2014 to develop a consensus neonatal AKI definition so that research and clinical studies would use the same definition making more studies comparable. Based upon information available at the time, the panel of neonatologists and nephrologists chose the modified neonatal Kidney Disease and Improving Global Outcomes (KDIGO) AKI definition based on staged changes in serum creatinine and urine output.

Current diagnostic methods for AKI are inadequate. There are several significant problems associated with the use of serum creatinine to diagnose AKI in premature infants despite it being the most accepted method currently available. The primary problem is that a rise in serum creatinine only occurs after as much as 50% of kidney function has been lost. In combination with the other known difficulties with serum creatinine (impacted by gender, BMI, ethnicity and chronic illnesses), it is not an ideal or useful biomarker for AKI.

Metabolomics is an emerging tool to define and detect clinical pathology with precision. With this technique, metabolites produced via cellular processes are identified and quantified in various biologic components including serum and urine using liquid chromatography-mass spectrometry. Metabolomics is a valuable tool to measure kidney function changes because the direct biproduct from the organ—urine—is easily and noninvasively collected and because the kidney is a very metabolically active organ continuously filtering, secreting, and excreting many different molecules.

The metabolomic methodology can be particularly useful in identifying if differences exist between stressed and non-stressed states and whether changes in metabolites are occurring earlier or differently than standardly measured biomarkers. A previous study demonstrated the potential to distinguish differences in the urinary metabolome between preterm infants who developed surgical necrotizing enterocolitis versus those who do not, but additional studies evaluating differences in preterm infants who develop acute kidney injury (AKI) are lacking (Moschino L, Verlato G, Stocchero M, Giordano G, Pirillo P, Meneghelli M, et al. Metabolomic analysis to predict the onset and severity of necrotizing enterocolitis. BMC Gastroenterol 2024, 24(1): 380).

What is needed are new biomarkers for AKI that are sensitive and specific for detecting AKI.

### BRIEF SUMMARY

In an aspect, a method of determining a subject has AKI comprises providing a urine sample from the subject, and detecting an AKI metabolomic biomarker in the urine sample.

In another aspect, a method of evaluating the effect of an AKI therapy in a subject with AKI comprises administering the AKI therapy to the subject, providing a urine sample from the subject after the administering, and detecting an AKI metabolomic biomarker in the urine sample.

In yet another aspect, a method of identifying metabolomic biomarkers associated with acute kidney injury (AKI) in preterm neonates comprises collecting urine samples from a population of preterm neonates, the population comprising a first subpopulation having no evidence of AKI, and a second subpopulation having AKI; performing a metabolomic analysis on the urine samples; comparing the first population and the second population and identifying metabolites significantly higher or significantly lower in the second subpopulation compared to the first subpopulation; and validating the metabolites to provide the metabolomic biomarkers.

### BRIEF DESCRIPTION OF THE DRAWINGS

FIG. 1 is a schematic of the liquid chromatography-mass spectroscopy (LC-MS) metabolomic analysis workflow.

FIG. 2 is a volcano plot of metabolites by AKI status including all urinary samples corrected for age and sex confounders and excluding control group. This volcano plot shows the fold change along the X axis and the statistical significance along the Y axis. The open circles are metabolites that were increased in abundance in the AKI group and the open triangles were decreased in abundance in the AKI group. Individuals in the control group 6, were removed from this analysis.

FIG. 3 is a volcano plot of metabolites by AKI status including only urinary samples prior to AKI for the AKI group, corrected for age and sex confounders and excluding control group. This volcano plot shows the fold change along the X axis and the statistical significance along the Y axis. The open circles are metabolites that were increased in abundance in the AKI group and the triangles were decreased in abundance in the AKI group. Individuals in the control group 6, were removed from this analysis.

FIG. 4 shows birth weight and average biomarker level.

FIG. 5 shows gestational age and average biomarker level.

FIG. 6 shows average biomarker median and deviation between AKI and no AKI groups.

The above-described and other features will be appreciated and understood by those skilled in the art from the following detailed description, drawings, and appended claims.

### DETAILED DESCRIPTION

Described herein is an approach to determine metabolomic biomarkers for AKI and the metabolomic biomarkers identified using this approach. A preferred metabolomic biomarker for AKI, particularly in infants born prematurely, would be easy to obtain, sensitive, specific for diagnosing AKI and would also allow clinicians to diagnose AKI as it is occurring when damage may be reversible instead of 12-24 hours after irreversible damage has occurred. Advantageously, unlike a single proteomic biomarker, a set of metabolomic biomarkers that can accurately detect AKI would be a significant improvement. Prior to the present disclosure, limited progress has been made in developing treatments or assessing how changes in current therapies affect AKI progression. The metabolomic biomarkers described herein can address some of these issues. Specifically, the metabolomic biomarkers described herein can be used to affect how AKI is diagnosed, categorized and treated.

Metabolomics is the study of the metabolome, a global collection of <1500 Dalton molecules present in every cell or biologic specimen. Metabolomics goes beyond the typical biomarker approach and analyzes metabolite levels that reflect multiple levels of cellular function, diet, environment, development and the microbiome. The metabolomic approach is a good fit for analyzing kidney function because the kidney is a highly metabolic organ and is responsible for continuously filtering, secreting and excreting multiple molecules. If the kidney becomes injured, the metabolites present in the blood and in the urine change significantly.

Described herein is a prospective observational study in the NICU wherein urine samples were collected from 40 preterm neonates, 5 who developed AKI, and 35 who did not. A metabolomic analysis was performed and in initial results up to 20 metabolites that are either significantly higher or significantly lower in the AKI group compared to the no AKI group after performing normalization were identified.

In an aspect, a method of identifying metabolomic biomarkers associated with acute kidney injury (AKI) in preterm neonates comprises collecting urine samples from a population of preterm neonates, the population comprising a first subpopulation having no evidence of AKI, and a second subpopulation having AKI; performing a metabolomic analysis on the urine samples; comparing the first population and the second population and identifying metabolites significantly higher or significantly lower in the second subpopulation compared to the first subpopulation, and validating the metabolites to provide the metabolomic biomarkers.

In an aspect, the preterm neonates are born at less than 32 weeks gestation.

In another aspect, the metabolomic analysis is done by liquid chromatography mass spectrometry (LC-MS). In an aspect, the metabolites are validated as AKI metabolomic biomarkers by correlation with proteomic biomarkers of kidney injury such as NGAL and/or performing a second study with a new population of AKI babies and see if they are again elevated.

In an aspect, the AKI metabolite is in one of the following pathways: the carnitine synthesis pathway, the oxidation of branched chain fatty acids, the fatty acid metabolism pathway, the beta oxidation of very long chain fatty acids, the mitochondrial beta-oxidation of short chain saturated fatty acids, the mitochondrial beta-oxidation of long chain saturated fatty acids, the citric acid cycle, the Warburg effect, the transfer of acyl groups to mitochondria, the tyrosine metabolism pathway, the phospholipid biosynthesis pathway, the tryptophan metabolism pathway, the glutamate metabolism pathway, the valine/leucine/isoleucine degradation pathway, the glutathione metabolism pathway, the ketone body metabolism pathway, the butyrate metabolism pathway, the mitochondrial electron transport chain, the phytanic acid peroxisomal oxidation pathway, the propanoate metabolism pathway, the vitamin K metabolism pathway, the arginine and proline metabolism pathway, the phenylacetate metabolism pathway, the beta-alanine metabolism pathway, the catecholamine biosynthesis pathway, the urea cycle, the ammonia recycling pathway, the amino sugar metabolism pathway, the cysteine metabolism pathway, the alanine metabolism pathway, the glucose-alanine cycle, the purine metabolism pathway, the glycine and serine metabolism pathway, the pyrimidine metabolism pathway, the nicotinate and nicotinamide metabolism pathway, the aspartate metabolism pathway, the lysine degradation pathway, the folate metabolism pathway, the arachidonic acid metabolism pathway, the malate-aspartate shuttle pathway, the phenylalanine and tyrosine metabolism pathway, the histidine metabolism pathway, glycolysis, the pyruvate metabolism pathway, gluconeogenesis, the pyruvaldehyde degradation pathway, the pantothenate and CoA biosynthesis pathway, the porphyrin metabolism pathway, the riboflavin metabolism pathway, the thiamine metabolism pathway, the bile acid biosynthesis pathway, the spermidine and spermine biosynthesis pathway, the caffeine metabolism pathway, and combinations thereof.

As used herein, the term “biomarker” generally refers to a molecule that is differentially present in a sample (e.g., urine) taken from a subject of one phenotypic status (e.g., having AKI) as compared with another phenotypic status (e.g., not having AKI). A biomarker is differentially present between different phenotypic statuses if the mean or median level of the biomarker in a first phenotypic status relative to a second phenotypic status is calculated to represent statistically significant differences. Exemplary tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest.

Exemplary AKI metabolomic biomarkers include furosemide, acesulfame, hexaethylene glycol, kynurenic acid, kynurenine, acetaminophen glucuronide, ceftazidime, fluconazole, ampicillin, DL-carnitine, tyramine, decanoylcarnitine, bilirubin, propionylcarnitine, cyclo(Pro_Tyr), tryptophan betaine, cyclo(Pro_Leu/Ile), cyclo(Pro_Val), 1, 2, 3, -propanetricarboxylic acid, 3-indoxyl sulfate, norfentanyl, panthenol/pantothenol, Val-Phe, 2′, 3′-cyclic GMP/3′,5′-cyclic GMP, DL-cystine, methylxanthine, DL-DOPA, pantothenic acid, acetylcholine, S-adenosylmethionine, succinic acid, dehydroascorbic acid, homovanillic acid, DL-glutamic acid, L-tyrosine, citric acid, pyroglutamic acid, pyruvic acid, L-tryptophan, choline, methylmalonic acid, L-valine, phenylacetylglutamine, ornithine, creatine, 3-ureidopropionic acid, panthenoic acid, carnosine, urocanic acid, N-acetylneuraminic acid, xanthosine, inosine, hypoxanthine, uric acid, xanthine, betaine, uridine, orotic acid, nicotinamide, N-Acetyl-L-aspartic acid, phenylalanine, riboflavin, thiamine, glycocholic acid, 5′-methylthioadenosine, 1,3,7-trimethyluric acid, theobromine, caffeine, paraxanthine, theophylline, 4-hydroxyphenylacetic acid, 2-furoylglycine, Asp_Glu, 2-aminoadipic acid, proline, sebacic acid, 2-furoic acid, 4-acetamidobutanoic acid, mandelic acid, 3,5-dimethoxybenzoic acid, N-acetylputrescine, N-acetyl-L-tyrosine, methylsuccinic acid, azelaic acid, labetalol, terephthalic acid, DL-stachydrine, metoclopramide, 2-aminoadipic acid, 4-acetamidobutanoic acid, hippuric acid, phthalic acid, Ala-Pro/Pro-Ala, hydroxyoctanoic acid, and combinations thereof.

In a specific aspect, the AKI metabolomic biomarkers comprise two or more of furosemide, acesulfame, terephthalic acid, DL-stachydrine, kynurenic acid, hexaethylene glycol, 2′3′-cyclic GMP, panthenol/pantothenol, fluconazole, Val-Phe, tryptophan betaine, 4-hydroxyphenylacetic acid, Cyclo (Pro_Leu/ile), tricarballylic acid, tyramine, decanoylcarnitine, norfentanyl, 3-indoxyl sulphate, cyclo (pro_Val), methylxanthine, DL-carnitine, 1,2,3-propanetricarboxylic acid, cyclo (Pro_Tyr), acetominophen, DL-dopa, and combinations thereof.

In an aspect, the AKI metabolomic biomarker comprises at least one elevated biomarker selected from furosemide, acesulfame, terephthalic acid, DL-stachydrine, kynurenic acid, hexaethylene glycol, 2′3′-cyclic GMP, panthenol/pantothenol, fluconazole, and Val-Phe; and at least one decreased biomarker selected from tryptophan betaine, 4-hydroxyphenylacetic acid, cyclo (Pro_Leu/ile), tricarballylic acid, tyramine, decanoylcarnitine, norfentanyl, 3-indoxyl sulphate, cyclo (pro_Val), methylxanthine, DL-carnitine, 1,2,3-propanetricarboxylic acid, cyclo (Pro_Tyr), acetominophen, and DL-dopa.

In an aspect, the at least one elevated biomarker comprises kynurenic acid and the at least one decreased biomarker comprises tryptophan betaine. In an aspect, elevated and decreased biomarkers can be determined relative to a normal population, and differences can be 1.5-fold, 2-fold, 3-fold, 4-fold, 5-fold, 6-fold, 7-fold, 8-fold, 9-fold, 10-fold, or more.

The use of multiple biomarkers increases the predictive value of the test and provides greater utility in diagnosis, patient stratification and patient monitoring. The process called “pattern recognition” detects the patterns formed by multiple biomarkers greatly improves the sensitivity and specificity of the diagnostic assay for predictive medicine. Subtle variations in data from clinical samples indicate that certain patterns of biomarkers can predict phenotypes such as the presence or absence of a certain disease, a particular stage of disease-progression, or a positive or adverse response to drug treatments. In aspect, 2, 3, 4, or more AKI metabolomic biomarkers are measured in a single assay.

In an aspect, a method of detecting an AKI metabolomic biomarker in a urine sample from a subject comprises providing the urine sample, and detecting an AKI metabolomic biomarker in the urine sample.

In an aspect, a method of determining a subject has AKI comprises providing a urine sample from the subject, and detecting an AKI metabolomic biomarker in the urine sample. Detecting the AKI metabolomic biomarker may be qualitative or quantitative as explained in detail below. In an aspect, an amount higher or lower than a threshold amount of AKI metabolomic biomarker indicates the subject has AKI.

In another aspect, a method of evaluating the effect of AKI therapy in a subject with AKI comprises administering the AKI therapy to the subject, providing a urine sample from the subject after the administering, and detecting an AKI metabolomic biomarker in the urine sample. Detecting the AKI metabolomic biomarker may be qualitative or quantitative as explained in detail below. In an aspect, an amount higher or lower than a threshold amount of AKI metabolomic biomarker indicates the efficacy of the AKI therapy.

In an aspect, the subject is an infant born prematurely, defined as 37 weeks or less of gestation, preferably 29 weeks or less of gestation. In another aspect, the subject is a neonate (<28 days old), an infant (0 days to 1 year old), a child or an adult who was born prematurely. In another aspect, the subject is a child or an adult suspected of having AKI.

In specific aspects, an amount greater than or less than a certain threshold amount may be used to diagnose AKI in a subject, predict in-hospital mortality of a subject suspected of having AKI, predict the need for in-hospital dialysis in a subject suspected of having AKI, determine whether a subject suspected of having AKI has transient or sustained AKI, determine the severity of the AKI in subject suspected of having AKI, determine the efficacy of a treatment for AKI, and/or determine the risk of an adverse event in a subject suspected of having AKI. The AKI metabolomic biomarker may exceed the threshold amount by about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50%, or about 75%, or about 100%, or more.

In embodiments, the AKI metabolomic biomarker and in particular the level of the biomarker(s) is measured on one or more occasions and an alteration in the levels as compared to normal reference levels over time is used as an indicator of AKI. The level of the biomarker(s) in a sample from a subject (e.g., urine) of a subject having AKI may be altered by as little as 10%, 20%, 30%, or 40%, or by as much as 50%, 60%, 70%, 80%, or 90% or more relative to the level of such biomarker(s) in a normal control. The level of the biomarker(s) in a sample from a subject (e.g., urine) of a subject having AKI may be altered by as little as 10%, 20%, 30%, or 40%, or by as much as 50%, 60%, 70%, 80%, or 90% or more relative to the level of such biomarker(s) in a previous sample from the subject. In embodiments, a subject sample is collected prior to the onset of symptoms of AKI. In embodiments, a subject sample is collected after the onset of symptoms of AKI. In embodiments, a subject sample is collected while the subject is undergoing treatment for AKI.

Accordingly, a biomarker profile may be obtained from a subject sample and compared to a reference biomarker profile obtained from a reference population, so that it is possible to classify the subject as belonging to or not belonging to the reference population. The correlation may take into account the presence or absence of the biomarkers in a test sample and the frequency of detection of the same biomarkers in a control. The correlation may take into account both of such factors to facilitate determination of AKI status.

In certain embodiments of the methods of qualifying AKI status, the methods further comprise managing subject treatment based on the status. Also included are methods where the biomarker(s) are measured again after subject management. In these cases, the methods are used to monitor the status of AKI, e.g., response to treatment, including improvement, maintenance, or progression of the disease.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

In an aspect, included herein are assays for detecting the AKI metabolomic biomarkers described herein. AKI metabolomic biomarkers can be detected using one or more methods well known in the art, including, without limitation, mass spectrometry, chromatography, spectroscopy (e.g., NMR), a chemical method, an immunoassay, and the like. In an aspect, the assay includes a reagent capable of detecting, binding or capturing the AKI metabolomic biomarker.

In embodiments, the AKI metabolomic biomarker(s) are detected using mass spectrometry. Mass spectrometry-based methods exploit the differences in mass of biomarkers to facilitate detection. Mass spectrometry can be combined with other assays, e.g., resolving the analyte in a sample by one or two passes through liquid or gas chromatography followed by mass spectrometry analysis. Methods for preparing a biological sample for analysis by mass spectrometry are well known in the art. Exemplary mass spectrometry technologies for use include, without limit, electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), electron impact ionization mass spectrometry (EI-MS), chemical ionization mass spectrometry (CI-MS), surface-enhanced laser desorption/ionization time-of-flight mass spectrometry (SELDI-TOF-MS), desorption/ionization on silicon (DIOS), secondary ion mass spectrometry (SIMS), quadrupole time of-flight (Q-TOF), atmospheric pressure chemical ionization mass spectrometry (APCI-MS), APCI-MS/MS, APCI(MS)11, atmospheric pressure photoionization mass spectrometry (APPI-MS), APPI-MS/MS, APPI-(MS), quadrupole, Fourier transform mass spectrometry (FTMS), ion trap, and hybrids of these methods, e.g., electrospray ionization quadrupole time-of-flight mass spectrometry (UPLC-ESI-QTOFMS) and two-dimensional gas chromatography electron impact ionization mass spectrometry (GCxGC-EI-MS).

The methods may be performed in an automated or semi-automated format. This can be accomplished, for example with MS operably linked to a liquid chromatography device (LC-MS/MS or LC-MS) or gas chromatography device (GC-MS or GC-MS/MS).

Other techniques for improving the mass accuracy and sensitivity of the MALDI-TOF MS can be used to analyze the analytes obtained on the collection membrane. These include the use of delayed ion extraction, energy reflectors and ion-trap modules. In addition, post source decay and MS-MS analysis are useful to provide further structural analysis. With ESI, the sample is in the liquid phase and the analysis can be by ion-trap, TOF, single quadrupole or multi-quadrupole mass spectrometers. The use of such devices (other than a single quadrupole) allows MS-MS or MSⁿ analysis to be performed. Tandem mass spectrometry allows multiple reactions to be monitored at the same time.

Capillary infusion may be employed to introduce the biomarker to a desired MS implementation, for instance, because it can efficiently introduce small quantities of a sample into a mass spectrometer without destroying the vacuum. Capillary columns are routinely used to interface the ionization source of a MS with other separation techniques including gas chromatography (GC) and liquid chromatography (LC). GC and LC can serve to separate a solution into its different components prior to mass analysis. Such techniques are readily combined with MS, for instance. One variation of the technique is that high performance liquid chromatography (HPLC) can now be directly coupled to mass spectrometer for integrated sample separation/and mass spectrometer analysis.

Quadrupole mass analyzers may also be employed. Fourier-transform ion cyclotron resonance (FTMS) can also be used for some embodiments. It offers high resolution and the ability of tandem MS experiments. FTMS is based on the principle of a charged particle orbiting in the presence of a magnetic field. Coupled to ESI and MALDI, FTMS offers high accuracy with errors as low as 0.001%.

In embodiments, an ion mobility spectrometer can be used to detect and characterize the biomarker(s). The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a biomarker or other substances in a sample. One advantage of ion mobility spectrometry is that it can operate at atmospheric pressure. In embodiments, the procedure is electrospray ionization quadrupole mass spectrometry with time of flight (TOF) analysis, known as UPLC-ESI-QTOFMS.

In embodiments, detection of the biomarker(s) involves use of chromatography methods that are well known in the art. Such chromatography methods include, without limit, column chromatography, ion exchange chromatography, hydrophobic (reverse phase) liquid chromatography, normal phase chromatography, hydrophilic interaction liquid chromatography, or other chromatography, such as thin layer, gas, or liquid chromatography (e.g., high-performance or ultraperformance liquid chromatography), or any combination thereof.

In embodiments, detection of the biomarker(s) involves use of spectroscopy methods that are well known in the art. Such chromatography methods include, without limitation, NMR, IR, and the like.

In embodiments, detection of the biomarker(s) involves use of immunoassays. In embodiments, the immunoassays involve the use of antibodies. Exemplary immunoassays include, without limitation, ELISA, flow chamber adhesion, colorimetric assays (e.g., antibody based colorimetric assays), biochip (e.g., antibody-based biochip), and the like.

Analytes (e.g., biomarkers) can be detected by a variety of detection methods. Detection methods may include use of a biochip array. Biochip arrays include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the biomarkers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

Another example is a barcode-style lateral flow immunoassay wherein antibodies and/or enzymes and other reagents begin absorbed into a porous plastic strip at certain positions, the familiar example being a home pregnancy test.

In an aspect, the assay includes a reagent capable of detecting, binding or capturing the AKI metabolomic biomarker. By “specifically binds” is meant an affinity agent (e.g., an antibody) that recognizes and binds a compound or agent of interest (e.g., a biomarker), but which does not substantially recognize and bind other molecules in a sample, for example, a biological sample.

The biomarkers and methods described herein can help identify infants who are either hospitalized or at home to improve kidney function and reduce morbidity and mortality. Serum creatine, for example, does not appear until 24 hours or more after developing AKI. The metabolites described herein could allow intervention much earlier and prior to significant kidney damage. In addition, the metabolites described herein could be used to customize therapeutic and dietary supplementation and prevent additional kidney damage.

In an aspect, detection of the biomarkers described herein can be combined with testing for neutrophil gelatinase-associated lipocalin (NGAL) test. Commercially available tests are the ProNephro AKI™ test and the NGAL Test™, marketed by BioPorto A/S. Both tests are particle-enhanced turbidometric immunoassays for quantitative determination of NGAL in human urine. The ProNephro AKI™ test is specifically designed for use in pediatric patients (3 months to 21 years).

In an aspect, detection of the biomarkers described herein can be combined with near-infrared spectroscopy (NIRS) monitoring to detect kidney hypoxia. NIRS is a non-invasive technique that can provide real-time information about renal tissue oxygen saturation, allowing for early diagnosis of kidney hypoxia. NIRS relies on a sensor attached to the skin that emits light in the near-infrared spectrum (700-900 nm). NIRS measures a mixture of arterial and venous capillary oxygenation. In the first two weeks of age average kidney oxygenation is typically >50%. Previous work has demonstrated that greater time with kidney hypoxia increases the risk of AKI. Without being held to theory, it is believed that if kidney oxygen drops below 50% and urinary biomarkers are collected at that time and those are abnormal, that there is a very high likelihood of AKI.

Exemplary AKI treatments include peritoneal dialysis, continuous kidney support therapy, theophylline, caffeine, diuretics such as furosemide or bumetanide, avoidance of nephrotoxic medications, and combinations thereof.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition, e.g., neoplasia.

As used herein, the terms “treat,” treating,” “treatment,” and the like refer to reducing or ameliorating a disease or condition, e.g., neoplasia, and/or symptoms associated therewith. It will be appreciated that, although not precluded, treating a disease or condition does not require that the disease, condition, or symptoms associated therewith be completely eliminated.

In an aspect, also included herein are articles for detecting the AKI metabolomic biomarkers in urine samples. Exemplary articles include a diaper, a foley catheter or collection bag, a lateral flow test, an absorbent cotton pad such as an ‘OB sponge’, and the like. The articles would include a reagent for detection of an AKI metabolomic biomarker.

For example, a diaper may include a strip with a reagent that changes color when urine comprises an AKI metabolomic biomarker. Similarly, a foley catheter could include a patch or strip that changes color when urine comprises an AKI metabolomic biomarker.

The invention is further illustrated by the following non-limiting examples.

### EXAMPLES

### Example 1: Identification of Urinary Metabolites in Preterm Neonates

### Methods

A prospective observational study of preterm neonates born <32 week gestational age was performed from 2021-2022. Participants were staged for AKI using the modified KDIGO neonatal definition including urine output. The patient demographics are in Table 1.

TABLE 1
Patient Demographics

| | No AKI (n = 27) | | AKI (n = 5) | | p-value |
|---|---|---|---|---|---|
| Gestational age | 29 | (27-30) | 27 | (25-29) | 0.13 |
| (weeks), mean SD | | | | | |
| Birth weight (g), | 1250 | (1020-1400) | 870 | (570-1075) | <0.01 |
| mean (SD) | | | | | |
| Sex, Female n (%) | 13 | (37) | 3 | (60) | 0.63 |

The primary outcome measure of this study was to identify differences in metabolomic biomarkers between participants with and without AKI. AKI was defined by the modified neonatal KDIGO definition including both serum creatinine and urine output (Table 2). All preterm neonates receive a basic metabolic profile (BMP) during their first 24 hours of life, which includes sCr level to monitor for kidney injury and electrolyte imbalance. A second BMP is performed at 48-72 hours of life and again at 1 week of age. If a preterm neonate is found to be at risk of AKI within two to seven days of age, sCr levels are checked more frequently. After 1 week of age, additional sCr are performed at the discretion of the attending physician. The neonate's diapers are weighed every 6 hours to determine urine output.

TABLE 2
KDIGO neonatal AKI diagnostic criteria

| AKI | | Urine output criteria |
|---|---|---|
| stage | SCr criteria | (hourly rate) |
| 0 | No change in SCr or SCr rise <0.3 mg/dL | ≥0.5 mL/kg/h |
| 1 | SCr rise ≥0.3 mg/dL rise within 48 h or | <0.5 mL/kg/h × 6-12 h |
| | SCr rise ≥1.5-1.9 × baseline SCr | |
| 2 | SCr rise ≥2.0-2.9 × baseline SCr | <0.5 mL/kg/h for >12 |
| | | h |
| 3 | SCr rise ≥3 × baseline SCr or SCr ≥2.5 | <0.3 mL/kg/h for ≥24 |
| | mg/dL or kidney support utilization. | h or anuria for ≥12 h |

Urine was collected approximately every six hours by cotton in the participants' diapers. A syringe was used to extract the urine into 0.5 mL plastic tubes in triplicate. The samples were then immediately placed in a −20° C. freezer. Between 24-72 hours the samples were transferred to and stored in a −80° C. freezer to prevent degradation.

All samples were maintained at −80° C. until the day of analysis. Due to the large number of samples present in the study, samples were analyzed in 8 batches. To mitigate analytical bias in downstream analysis, samples were distributed into batches in a manner that ensured equivalent distributions of sex, gestational age, and AKI status, then samples were randomized within batch to further reduce bias. To limit the analysis time between batches, and preserve sample integrity, each batch was prepared immediately prior to the end of the previous batch in order to keep the instrument running continuously throughout all 8 batches.

Pooled urine (provided by collaborator, 14 aliquots) was thawed and combined, then centrifuged for 5 minutes at 1,500×g at 4° C. and distributed into twelve 50 L aliquots to serve as quality control samples. Additionally, a pooled urine dilution curve was prepared by diluting pooled urine with chilled water to 25% concentration, 50% concentration, and 75% concentration.

For each batch, samples and pooled urine were removed from the −80° C. freezer and thawed at 4° C. Samples were then centrifuged for 1 min at 12,000×g at 4° C. to remove any particulates. 50 μL of each sample was aliquoted into a glass-lined 96-well plate (Thermo Scientific, Part Number: 60180-P332). The samples and 96-well plate were maintained on ice prior to being placed into the autosampler for analysis. A silicone mat with slits (Thermo Scientific, Part Number: 60180-M112) was used to cover the wells in order to prevent evaporation. After running, plates were sealed with foil covers (Research Products International, Part Number: ZC2007-24) and stored at −80° C.

Sample analysis was performed using a method known in the art. Briefly, samples were separated using a Waters™ Acquity UPLC HSS T3 column held at 40° C. (150 mm×1 mm×1.8 m particle size; Waters). 1 μL of sample was injected on a Vanquish™ Neo System (Thermo Scientific) followed by separation with the following gradient at 70 L/min flow rate: initial conditions of 100% Mobile phase A (0.1% formic acid in water) for 0.5 min, then linear increase to 100% Mobile phase B (0.1% formic acid in 95% methanol) over the next 8 min. 100% Mobile phase B was maintained for 1 min before returning to 0% mobile phase B over the next 0.75 min and equilibrating at 0% Mobile phase B for a remaining 1.25 min.

The LC system was coupled to a Q Exactive™ HF Orbitrap mass spectrometer through a heated electrospray ionization (HESI II) source (Thermo Scientific). Source conditions were as follows: HESI II probe at 30° C., capillary temperature at 300° C., sheath gas flow rate at 30 units, aux gas flow rate at 10 units, sweep gas flow rate at 1 units, spray voltage at 13.2 kVJ for positive and negative mode, and S-lens RF at 50.0 units. The MS was operated in a polarity switching mode acquiring positive and negative full MS and MS2 spectra (Top2) within the same injection. Acquisition parameters for full MS scans in both modes were 30,000 resolution, 1×10⁶ automatic gain control (AGC) target, 100 ms ion accumulation time (max IT), and 70 to 750 m/z scan range. MS2 scans in both modes were then performed at 30,000 resolution, 1×105 AGC target, 50 ms max IT, 1.0 m/z isolation window, stepped normalized collision energy (NCE) at 20, 30, 40, and a 10.0 s dynamic exclusion.

The resulting LC-MS data were processed using Compound Discoverer 3.3 (Thermo Scientific). All peaks with a 0 min to 11.5 min retention time and 0 Da to 5000 Da MS1 precursor mass were aggregated into distinct chromatographic profiles (i.e., compound groups) using a 10-ppm mass and 0.5 min retention time tolerance. Profiles not reaching a minimum peak intensity of 1×10⁶, a maximum peak-width of 1, and a signal-to-noise (S/N) ratio of 3 were excluded from further processing. MS/MS spectra were searched within Compound Discoverer using mzCloud and mzVault spectral libraries. Annotations were assigned using a mass tolerance of 10 ppm. Data were filtered in Compound Discover requiring each feature to be present in at least 100 of the samples with a Peak Rating Threshold of at least 5. Gap filling was performed with a mass tolerance of 10 ppm and a S/N threshold of 1.5. The resulting features were manually checked for quality, and putative identifications from the Compound Discover Compound table were used to annotate these features. Provided annotations are Level 1 or Level 2 annotations as known in the art. Metabolite features were removed if % RSD of replicate quality control was >30%.

The resulting data set was analyzed using Metaboanalyst 5.0 (Pang 2021) and with R statistical and plotting environment (v 4.4.0). Effect of AKI was evaluated using mixed effect linear models to account for account for confounding variables of sex and age and repeated measures within individuals. Significance was determined by model comparison of null model without the AKI variable using the gamlssMX( ) function in the gamlss.mx package in R, which returns the log-likelihood ratio and significance. Resulting p-values were adjusted for multiple hypothesis testing using the ‘fdr’ method in the p.adjust( ) function.

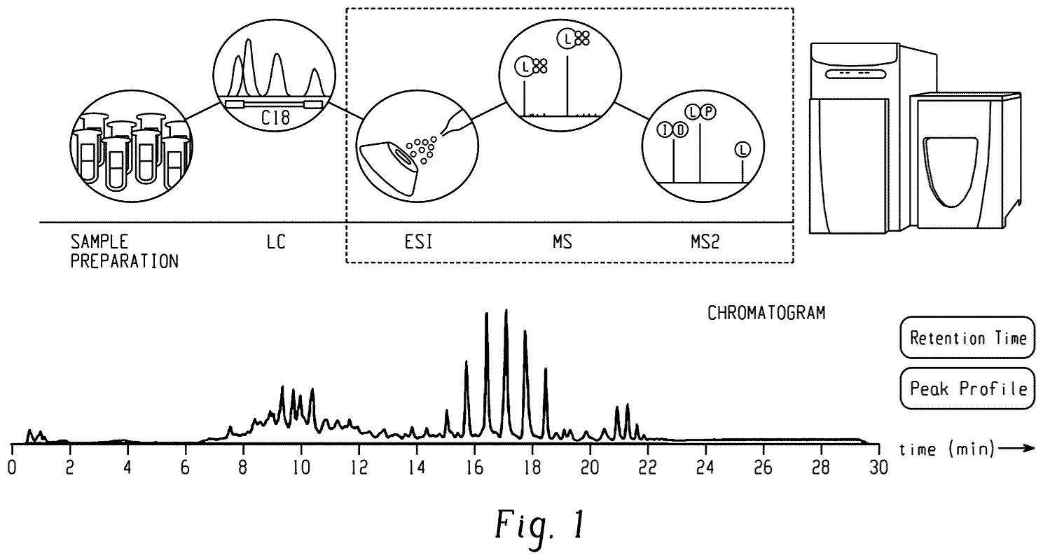

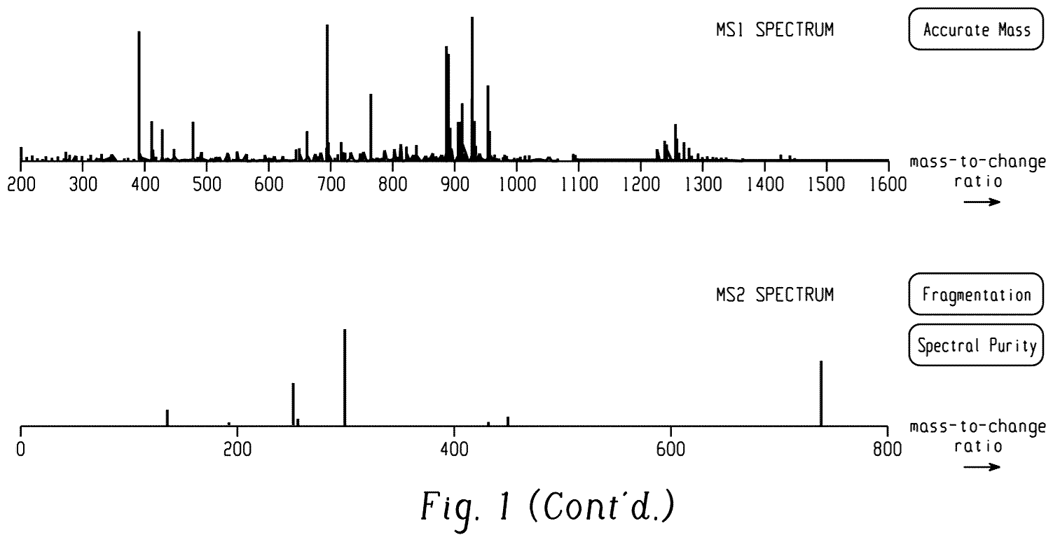

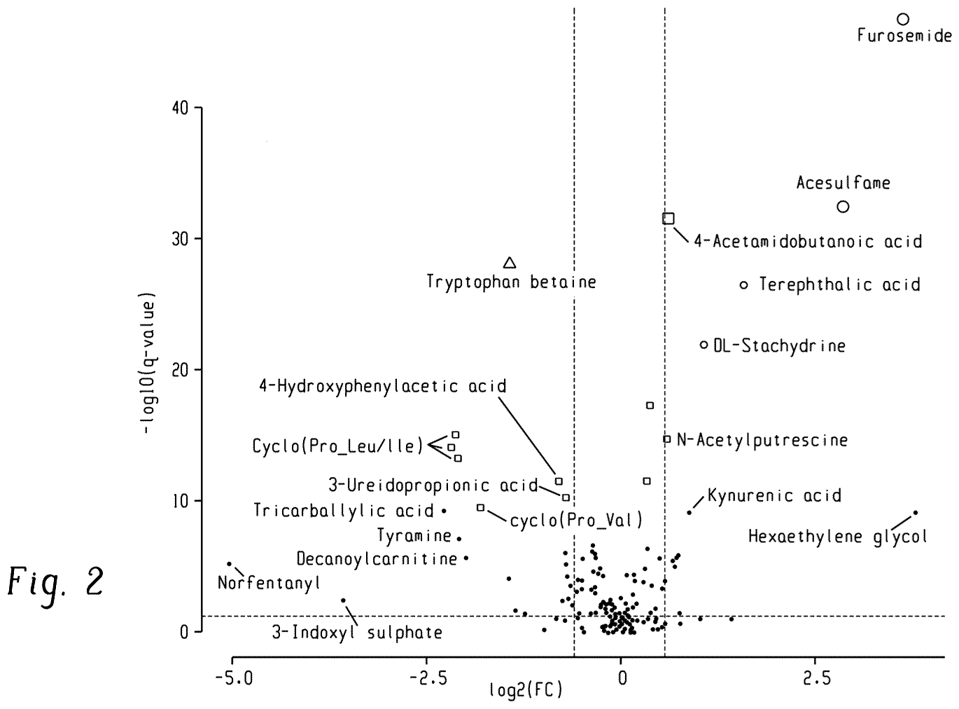

The metabolomic workflow is illustrated in FIG. 1.

### Results

40 subjects were enrolled, 32 included in the analysis while eight were utilized as controls. Of the 32 analyzed subjects, five were diagnosed with AKI (13%). Of the five with AKI, four had stage 1 while 1 had stage 2. Four subjects were diagnosed with serum creatinine criteria while one subject with urine output criteria. Demographics of neonates with and without AKI can be seen in Table 1. Neonates with AKI were more likely to be lower birth weight (850 vs. 1250 g, p<0.01).

Initial metabolomic analysis with PCA demonstrates significant overlap between AKI and no AKI groups (data not shown).

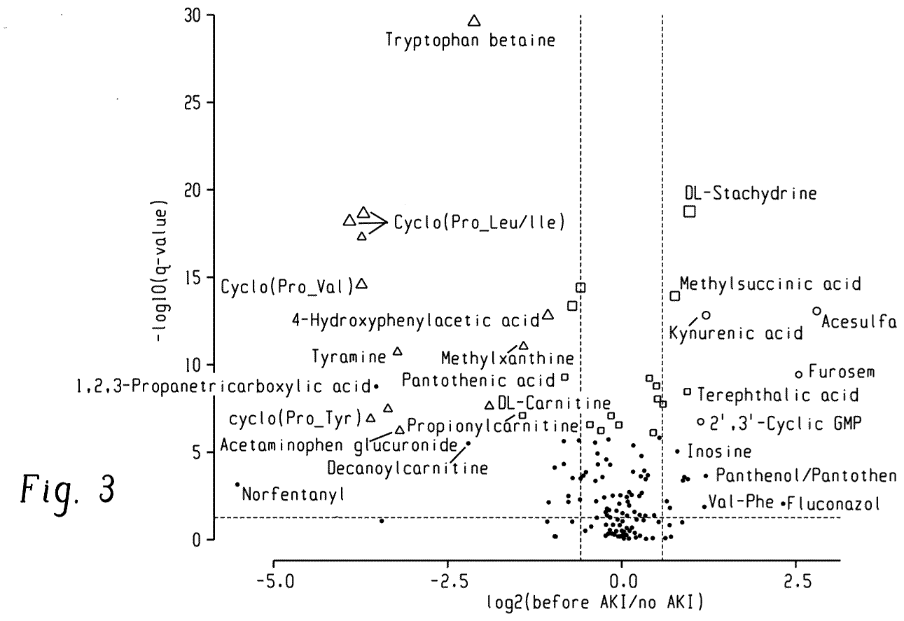

The next step of metabolomic analysis by volcano plot shows multiple candidate metabolites with differences between the AKI and no AKI groups when analyzing all the urinary samples, including before and after AKI in that group, and correcting for gestational age and sex excluding the control group (FIG. 3). Statistically significant elevations in Furosemide, Acesulfame, Terephthalic acid, DL-Stachydrine, Kynurenic acid, and Hexaethylene glycol are noted (Table 3). Statistically significant decreases in Tryptophan betaine, 4-Hydroxyphenylacetic acid, Cyclo (Pro_Leu/ile), Tricarballylic acid, Tyramine, Decanoylcarnitine, Norfentanyl, and 3-Indoxyl sulphate are noted (Table 3).

TABLE 3
AKI vs. No AKI

| | Adjusted | Log2(AKI/ |
|---|---|---|
| Metabolites | p-value | noAKI) |
| Norfentanyl | 4.97E−06 | −5.030199336 |
| 3-Indoxyl sulphate | 0.003433163 | −3.558958417 |
| 1,2,3-Propanetricarboxylic acid | 4.25E−10 | −2.283617234 |
| Cyclo(Pro_Leu/Ile), 5.915 | 6.34E−15 | −2.175958634 |
| Cyclo(Pro_Leu/Ile), 5.645 | 6.74E−16 | −2.131614847 |
| Cyclo(Pro_Leu/Ile), 5.768 | 4.41E−14 | −2.089227543 |
| Tyramine | 7.14E−08 | −2.071977829 |
| Decanoylcarnitine | 2.08E−06 | −1.980857967 |
| cyclo(Pro_Val) | 2.67E−10 | −1.78748428 |
| cyclo(Pro_Tyr) | 7.12E−05 | −1.427101778 |
| Tryptophan betaine | 6.68E−29 | −1.405272797 |
| Bilirubin | 0.021764235 | −1.337959672 |
| Metoclopramide | 0.045302523 | −1.227284263 |
| 4-Hydroxyphenylacetic acid | 2.39E−12 | −0.790051336 |
| DL-Dopa | 0.003393612 | −0.735869118 |
| Propionylcarnitine | 7.91E−07 | −0.709629732 |
| 3-Ureidopropionic acid | 4.46E−11 | −0.70454309 |
| DL-Carnitine | 5.64E−06 | −0.699133227 |
| 2-Aminoadipic acid | 4.29E−05 | −0.677992825 |
| Methylxanthine, 3.309 | 0.002350116 | −0.669071849 |
| 2-Furoylglycine | 0.000242936 | −0.629485292 |
| DL-Glutamic acid | 0.0064416 | −0.616222592 |
| Ornithine | 0.000647111 | −0.552785729 |
| Asp_Glu | 8.97E−05 | −0.536600586 |
| DL-Cystine | 0.036962635 | −0.526223898 |
| Glycocholic acid | 1.96E−06 | 0.50556211 |
| Azelaic acid | 0.000405947 | 0.55714965 |
| Inosine | 0.000130764 | 0.58501971 |
| N-Acetylputrescine | 1.77E−15 | 0.607099926 |
| 4-Acetamidobutanoic acid | 2.16E−32 | 0.614906853 |
| Hippuric acid | 3.14E−06 | 0.686116788 |
| Phthalic acid | 8.72E−06 | 0.70378872 |
| 2′,3′-Cyclic GMP/3′,5′-Cyclic GMP | 1.73E−06 | 0.735766527 |
| 3,5-Dimethoxybenzoic acid | 1.31E−06 | 0.747367794 |
| Ala-Pro/Pro-Ala | 0.031143105 | 0.759801128 |
| Hydroxyoctanoic acid, 6.265 | 0.030058785 | 0.771986234 |
| Kynurenic acid | 6.11E−10 | 0.890235969 |
| DL-Stachydrine | 1.08E−22 | 1.093306143 |
| Terephthalic acid | 2.31E−27 | 1.598824284 |
| Acesulfame | 2.88E−33 | 2.879952307 |
| Furosemide | 1.36E−47 | 3.653118089 |
| Hexaethylene glycol | 6.91E−10 | 3.814957203 |

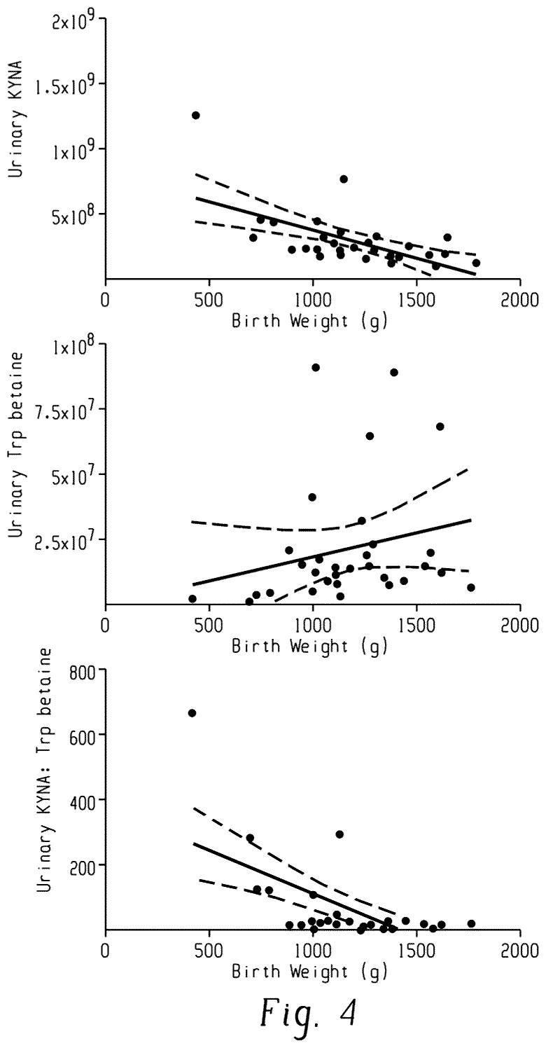

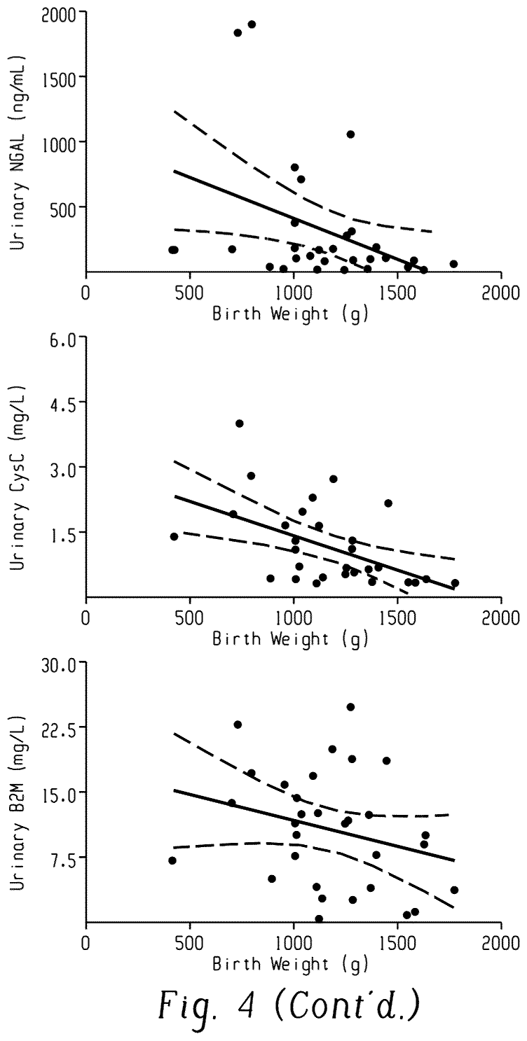

The final metabolomic analysis by volcano plot shows similar but different candidate metabolites with differences between the AKI and no AKI groups when analyzing just the urinary samples before the diagnosis of AKI in that group compared to the no AKI group, also correcting for gestational age and sex excluding the control group (FIG. 4). Statistically significant elevations in Acesulfame, Kynurenic acid, Furosemide, 2′3′-Cyclic GMP, Panthenol/Pantothenol, Fluconazole, and Val-Phe are noted (Table 4). Statistically significant decreases in Tryptophan betaine, Cyclo (Pro_Leu/Ile), Cyclo (pro_Val), Methylxanthine, Tyramine, DL-Carnitine, 1,2,3-Propanetricarboxylic acid, cylco (Pro_Tyr), Acetominophen, Decanoylcarnitine, Norfentanyl, and DL-Dopa are noted (Table 4). Of note, the markers identified in FIG. 4 are significant as they represent biomarkers identifiable before the diagnosis of disease.

TABLE 4
Before AKI vs. No AKI

| | Adjusted | Log2(beforeAKI/ |
|---|---|---|
| Metabolites | p-value | noAKI) |
| Norfentanyl | 0.00078247 | −5.510471796 |
| Cyclo(Pro_Leu/Ile), 5.915 | 5.81E−19 | −3.91632932 |
| cyclo(Pro_Val) | 2.53E−15 | −3.754028536 |
| Cyclo(Pro_Leu/Ile), 5.768 | 4.60E−18 | −3.728939013 |
| Cyclo(Pro_Leu/Ile), 5.645 | 1.97E−19 | −3.716438477 |
| cyclo(Pro_Tyr) | 1.32E−07 | −3.599543635 |
| 1,2,3-Propanetricarboxylic acid | 3.25E−08 | −3.356843734 |
| Tyramine | 1.81E−11 | −3.21920681 |
| Acetaminophen glucuronide | 6.15E−07 | −3.182527478 |
| Decanoylcarnitine | 3.95E−06 | −2.211238924 |
| Tryptophan betaine | 2.28E−30 | −2.133299708 |
| DL-Carnitine | 2.01E−08 | −1.876435285 |
| Propionylcarnitine | 6.46E−08 | −1.439168027 |
| Methylxanthine, 3.309 | 9.24E−12 | −1.40003873 |
| 4-Hydroxyphenylacetic acid | 1.52E−13 | −1.081740464 |
| DL-Dopa | 0.008145769 | −1.056920548 |
| DL-Glutamic acid | 8.29E−05 | −0.972133243 |
| Pantothenic acid | 6.05E−10 | −0.850218799 |
| 2-Furoylglycine | 5.57E−05 | −0.841788309 |
| Asp_Glu | 2.63E−06 | −0.831722186 |
| 2-Aminoadipic acid | 0.003807364 | −0.778649782 |
| Succinic acid | 0.008694297 | −0.770006898 |
| Methylxanthine, 3.509 | 0.000337342 | −0.719014948 |
| Proline | 4.17E−14 | −0.717433916 |
| 3-Ureidopropionic acid | 1.05E−07 | −0.698256625 |
| Ornithine | 0.000395897 | −0.618333882 |
| Sebacic acid | 2.36E−06 | −0.615108846 |
| Methylmalonic acid | 3.73E−15 | −0.605508357 |
| Theophylline | 0.006127492 | −0.604317227 |
| 2-Furoic acid | 0.000395373 | −0.600449946 |
| Citric acid | 0.000256349 | −0.566367092 |
| Nicotinamide | 0.000167391 | −0.534971703 |
| 4-Acetamidobutanoic acid | 9.65E−09 | 0.512835713 |
| Mandelic acid | 1.85E−06 | 0.539885243 |
| 3,5-Dimethoxybenzoic acid | 0.026248662 | 0.553008803 |
| Ampicillin | 0.012572649 | 0.575807074 |
| N-Acetylputrescine | 2.01E−08 | 0.579257664 |
| Ceftazidime | 0.008100644 | 0.654332352 |
| N-Acetyl-L-tyrosine | 0.017885986 | 0.685761994 |
| Methylsuccinic acid | 1.37E−14 | 0.732724441 |
| Inosine | 1.05E−05 | 0.789266621 |
| Azelaic acid | 0.000459505 | 0.865710042 |
| Labetalol | 0.000354371 | 0.894198651 |
| S-Adenosylmethionine | 0.000418845 | 0.922850491 |
| Terephthalic acid | 3.06E−09 | 0.931053923 |
| DL-Stachydrine | 1.63E−19 | 0.950321299 |
| 2′,3′-Cyclic GMP/3′,5′-Cyclic GMP | 2.10E−07 | 1.115783581 |
| Val-Phe | 0.014862032 | 1.17466807 |
| Panthenol/Pantothenol | 0.000278419 | 1.187669649 |
| Kynurenic acid | 1.41E−13 | 1.19720283 |
| Fluconazole | 0.009169747 | 2.286795561 |
| Furosemide | 3.73E−10 | 2.528141689 |
| Acesulfame | 9.04E−14 | 2.780194873 |

### Example 2: Validation of Kynurenic Acid and Tryptophan Betaine as Urinary Biomarkers in Preterm Neonates

### Methods

Participants: Neonates born <32 weeks' gestational age (GA) were approached for informed parental consent. Exclusion of participants occurred if any of the following criteria were met: 1) enrollment and placement of NIRS sensors not possible by 96 hours of life, 2) non-English or non-Spanish speaking families, 3) refusal by attending physician, 4) mothers who could not participate in the consent process, 5) investigative team unable to consent parent, 6) NIRS monitors unavailable, 7) Documentation of congenital anomalies of the kidney or urinary tract.

### Urine Collection: See Example 1

Metabolomic Analysis: Urine samples were analyzed in 8 batches with equal participant distributions of sex, GA, and AKI status. Each batch was thawed to 4° C. and centrifuged for 1 minute at 12,000×g to remove particulates and then aliquoted into a glass-lined 96-well plate (Thermo Scientific, Part Number: 60180-P332). Samples were then placed in the autosampler for analysis. A pooled urine dilution curve was prepared by diluting pooled urine with chilled water. Samples were separated using a Waters™ Acquity UPLC HSS T3 column held at 40° C. Samples were injected on a Vanquish Neo System (Thermo Scientific) followed by separation. The liquid chromatography (LC) was coupled to a Q Exactive™ HF Orbitrap mass spectrometer through a heated electrospray ionization (HESI II) source (Thermo Scientific). The mass spectrometer was operated in a polarity switching mode acquiring positive and negative full mass spectrometry (MS) and tandem mass spectrometry (MS2) spectra (Top2) within the same injection. Data was processed using Compound Discover 3.3 (Thermo Scientific). MS/MS spectra were searched within Compound Discoverer using mzCloud and mzVault spectral libraries. Data were filtered in Compound Discover. Metabolite features were removed if % relative standard deviation of replicate quality control was >30%.

Proteomic Analysis: Proteomic analyses were completed at Cincinnati Children's Hospital Medical Center in Cincinnati, OH. Urinary NGAL was measured on a Siemens BNII Nephelometer using a particle-enhanced turbidimetric immunoassay run at a 5× dilution with a minimum detectable NGAL concentration of 46.9 ng/mL. Urinary CysC and B2M were measured on a Roche c 311 clinical chemistry analyzer. CysC concentrations were determined using a particle-enhanced turbidimetric immunoassay with a detection range of 0.4-6.80 mg/L. B2M concentration was measured using an immunoturbidimetric assay with a detection range of 0.2-63.8 mg/L. Some samples exceeded this range due to external dilutions accounting for low sample volumes.

Outcome measures: The primary outcome measure of this study was to identify differences in metabolomic and proteomic biomarkers between participants with and without AKI. AKI was defined by the modified neonatal KDIGO definition including both serum creatinine and urine output (Table 2). All preterm neonates received a basic metabolic profile (BMP) during their first 24 hours of life, which included sCr level to monitor for kidney injury and electrolyte imbalance. A second BMP was performed at 48-72 hours of life and again at 1 week of age. If a preterm neonate was found to be at risk of AKI within two to seven days of age, sCr levels were checked more frequently. After 1 week of age, additional sCr were performed at the discretion of the attending physician. The neonate's diapers were weighed every 6 hours to determine urine output.

The secondary outcome was to identify the relationship between metabolomic, proteomic biomarkers, and kidney oxygenation measured by NIRS. For all neonates in this study, kidney oxygenation was measured at the right and left flank using INVOS 5100 C (Somanetics, Troy, MI, USA) four channel NIRS monitors. NIRS sensors were placed over a Mepitel® adhesive dressing to protect the neonate's skin. The kidney sensors were placed without ultrasound guidance and were changed per the company's recommendation after 3-4 days of use. Sensors were also changed if the sensor was not reading accurately, or the signal integrity was disrupted. Tissue oxygenation was recorded every one to six seconds between two and fourteen days of age. Bilateral kidney oxygenation was recorded for two-six hours between the fifth and eighth days of age and again between the ninth and fourteenth days of life. This bilateral monitoring was completed by placing NIRS sensors on both the left and right flank simultaneously. Handling and positioning of the neonates was not restricted, and re-positioning occurred every three-six hours.

Statistical analysis: Demographic and clinical information was summarized using the median and interquartile range (IQR), or with frequencies and percentages. Area under each patient's profile of a given biomarker over time was calculated by the trapezoidal rule. This value was then divided by either the length of time the biomarker was measured, or up to the onset of AKI, whichever came earliest. Demographic and clinical characteristics, as well as average biomarker abundance, were compared between no AKI and AKI groups using Wilcoxon rank-sum tests or exact unconditional tests for categorical factors. Kidney hypoxia for each patient was summarized by the percentage of non-missing RrSO₂ readings that were <50% over the same time interval a patient's biomarker profile was observed. Associations between average biomarker and kidney hypoxia level were assessed using Kendall's tau. A p-value less than 0.05 was considered significant.

### Results and Discussion

Kynurenic acid (KYNA) is a byproduct of the kynurenine pathway (KP), responsible for tryptophan (Trp) catabolism, and a potential biomarker for AKI. The KP is activated by several enzymes, including idoleamine 2,3-dioxygenase (IDO), which is upregulated in periods of inflammation and acts as an immune regulator. This, coupled to the fact that kidney dysfunction may lead to the accumulation of Trp metabolites, makes KYNA an attractive candidate to diagnose AKI. Some human studies have already shown elevated urinary and serum KYNA indicates the presence and increased severity of AKI. Another urinary metabolite, tryptophan betaine (Trp betaine), a trimethylated derivative of Trp that is also known as hypaphorine, plays a role in reducing the presence of certain inflammatory cytokines that may activate enzymes responsible for KP initiation, and has been shown to reduce inflammation in human endothelial cells in vitro.

This example aims to assess several novel, non-traditional markers of AKI in neonates. The first aim was to determine the differences in urinary NGAL, CysC, B2M, KYNA, Trp, and the ratio of KYNA/Trp between AKI and non-AKI neonates. Without being held to theory, it was hypothesized that neonates with AKI would have higher urinary NGAL, CysC, B2M, KYNA, and KYNA/Trp, and lower Trp. The second aim was to identify the relationship between these biomarkers and RrSO₂ obtained via NIRS. Without being held to theory, it was hypothesized that there would be a direct relationship between RrSO₂ and urinary Trp betaine, and an inverse relationship between RrSO₂ and NGAL, CysC, B2M, KYNA, and KYNA/Trp betaine.

Study participants and demographics: 32 neonates enrolled in the study and five (16%) developed AKL. Neonates who developed AKI tended to weigh less at birth (p=0.01) and be smaller for GA (p 0.04) compared to the neonates who never developed AKI during the study period (Table 5). Neonates with AKI were diagnosed at a mean 241.4 hours of age. Urine collection began at a mean 87.8 hours of age.

TABLE 5
Selected Demographic data

| | Full Cohort | | No AKI | | AKI | | p |
|---|---|---|---|---|---|---|---|
| | (n = 32) | | (n = 27) | | (n = 5) | | value |
| Gestational age (weeks) | 29 | (27, 29) | 29 | (27, 30) | 27 | (24, 29) | 0.11 |
| Birth weight (g) | 1120 | (1010, 1360) | 1250 | (1020-1370) | 710 | (430-1010) | 0.01 |
| Sex, n (%) | | | | | | | 0.65 |
| Male | 17 | (53) | 15 | (56) | 2 | (40) | |
| Female | 15 | (47) | 12 | (44) | 3 | (60) | |
| Size, n (%) | | | | | | | 0.04 |
| Small for gestational age | 2 | (6) | 0 | | 2 | (40) | |
| Average for gestational | 28 | (88) | 25 | (93) | 3 | (60) | |
| age | | | | | | | |
| Large for gestational age | 2 | (6) | 2 | (7) | 0 | (0) | |

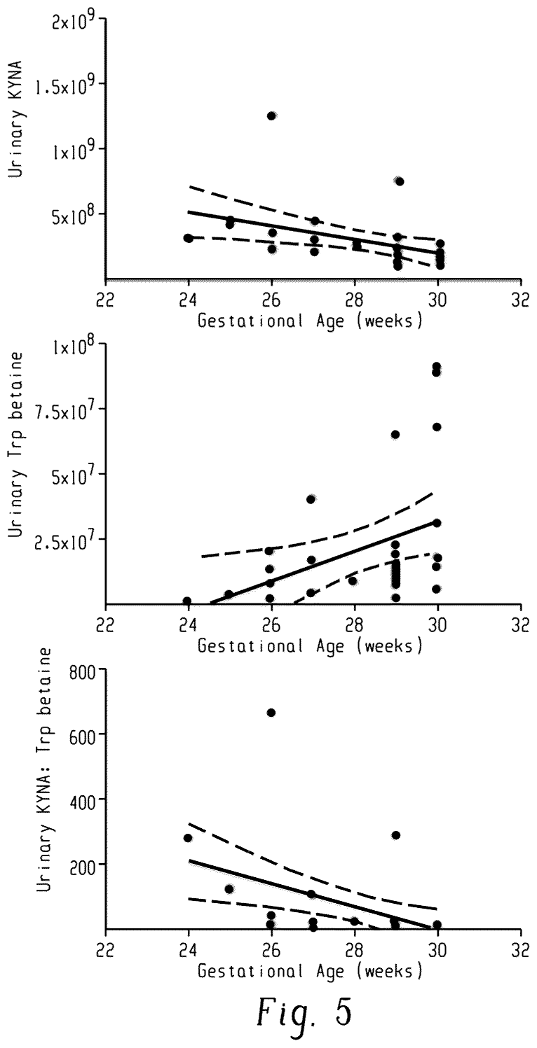

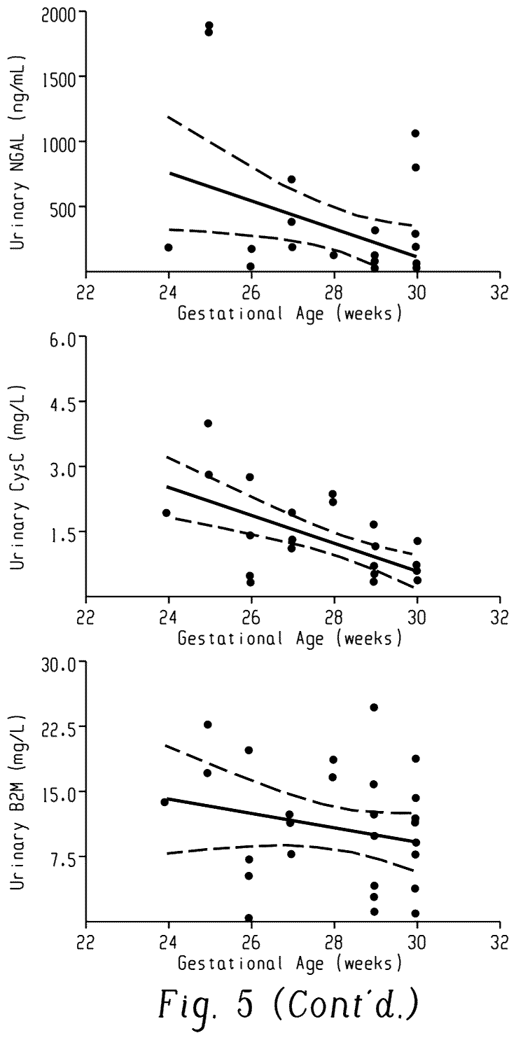

Urinary Biomarkers: KYNA, NGAL, and CysC were found to be negatively correlated with birth weight and gestational age (GA). Trp betaine was the only biomarker positively correlated with birth weight and GA (FIGS. 4, 5). Furthermore, a negative correlation between urinary KYNA and both birth weight and GA were observed, and a positive correlation between urinary Trp betaine and both birth weight and GA suggesting a component of developmental regulation.

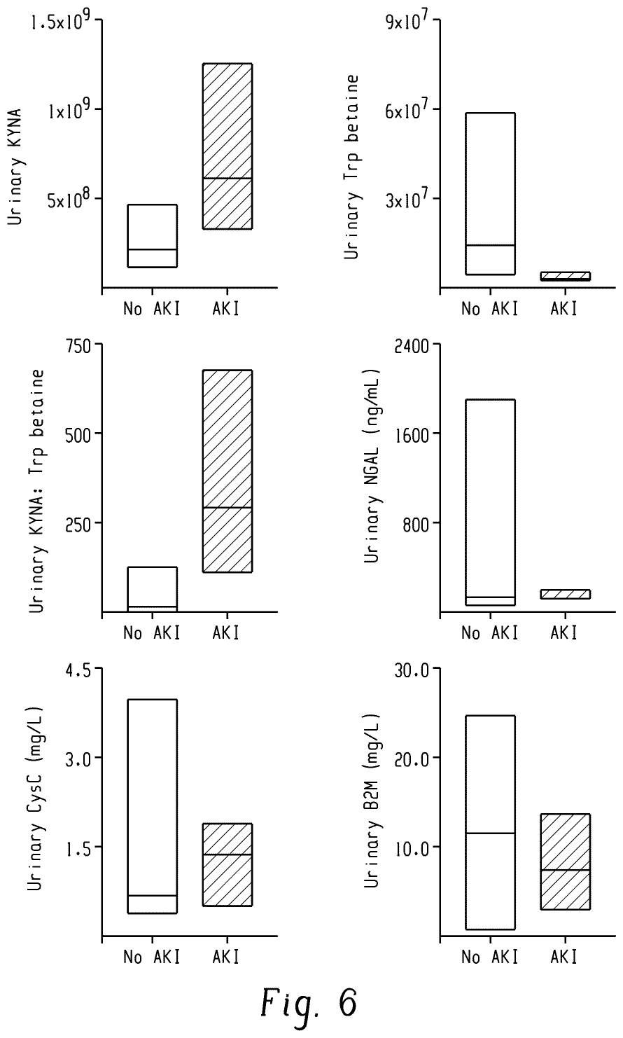

AKI and Urinary Biomarkers: Neonates with AKI had significantly higher average urinary KYNA (p=0.01) and average urinary ratio of KYNA:Trp betaine (p<0.01). Average values of urinary Trp betaine were significantly lower in neonates with AKI compared to those without AKI (p<0.01). There was no significant difference between AKI and no AKI groups for the NGAL, CysC, or B2M (p >0.30 for each) (Table 6, FIG. 6).

TABLE 6
Association between average biomarker level and AKI status

| | No AKI | AKI | Optimal | | | | auROC | |
|---|---|---|---|---|---|---|---|---|
| | average | average | cut-off | | | Youden | (95% | p |
| Biomarker | (median) | (median) | value | Sensitivity | Specificity | Index | CI) | value |
| KYNA | 2.25 × 10⁸ | 6.18 × 10⁸ | 3.385 × 10⁸ | 77.11 | 81.90 | 0.59 | 0.95 | 0.01 |
| | | | | | | | (0.76, | |
| | | | | | | | 0.99) | |
| Trp betaine | 1.56 × 10⁷ | 3.14 × 10⁶ | 7.073 × 10⁶ | 91.57 | 85.94 | 0.78 | 0.98 | <0.01 |
| | | | | | | | (0.82, | |
| | | | | | | | 0.99) | |
| KYNA: | 15.72 | 290.60 | 44.93 | 93.98 | 89.45 | 0.83 | 0.98 | <0.01 |
| Trp betaine | | | | | | | (0.94, | |
| | | | | | | | 0.99) | |
| NGAL | 138.40 | 201.10 | 48.38 | 80.25 | 49.86 | 0.30 | 0.58 | 0.679 |
| (ng/mL) | | | | | | | (0.36, | |
| | | | | | | | 0.77) | |
| CysC | 0.71 | 1.39 | 0.6675 | 67.90 | 63.09 | 0.31 | 0.64 | 0.393 |
| (mg/L) | | | | | | | (0.39, | |
| | | | | | | | 0.83) | |
| B2M | 11.62 | 7.55 | 13.32 | 78.75 | 32.82 | 0.12 | 0.65 | 0.376 |
| (mg/L) | | | | | | | (0.37, | |
| | | | | | | | 0.85) | |

Urinary Biomarkers and Kidney Oxygenation: The percentage of time over which urine samples were collected that an infant's RrSO₂ was <50% ranged from 0.8% to 62.5% with a median of 19% (IQR: 6-36%). Kidney hypoxia was typically positively correlated with urinary biomarker levels. Trp betaine was the only biomarker negatively correlated with kidney hypoxia. KYNA and CysC had τ values of 0.35 and 0.31 (both p<0.01), respectively, and Trp betaine had a τ value of −0.31 (p<0.01) (Table 7).

TABLE 7
Association between average biomarker
response and kidney hypoxia

| | Correlation with kidney hypoxia | | |
|---|---|---|---|
| Marker | | tau (95% CI) | p-value |

| | | | |
|---|---|---|---|
| KYNA | 0.35 | (0.11, 0.58) | 0.006 |
| Trp betaine | −0.31 | (−0.58, −0.04) | 0.015 |
| NGAL | −0.06 | (−0.35, 0.23) | 0.646 |
| CysC | 0.31 | (0.14, 0.48) | 0.014 |
| B2M | 0.12 | (−0.10, 0.34) | 0.333 |

### Discussion of Example 2

The objective was to examine urinary metabolomic and proteomic biomarkers in preterm neonates with AKI. Identification of urinary biomarkers that differ significantly between preterm neonates with and without AKI is a key step in developing new diagnostic criteria for AKI. A key finding is that preterm neonates with AKI had significantly higher average urinary KYNA and KYNA:Trp betaine and significantly lower average urinary Trp betaine than those without AKI. No significant difference between AKI and non-AKI preterm neonates was observed with urinary proteins NGAL, CysC, and B2M. Furthermore, a negative correlation between urinary KYNA and both birth weight and GA were observed, and a positive correlation between urinary Trp betaine and both birth weight and GA suggesting a component of developmental regulation. Finally, a correlation between kidney hypoxia and levels of KYNA, CysC, and Trp betaine was observed. These findings are an important step forward in utilizing urinary biomarkers in the early diagnosis of AKI in preterm neonates.

The metabolomic results, which indicated elevated urinary KYNA, KYNA: Trp betaine, and decreased urinary Trp betaine in preterm neonates with AKI, are consistent with previous research. A study by Arregar et al. found that elevated urinary KYNA predicted renal non-recovery in ICU patients with AKI (Aregger, F., Uehlinger, D. E., Fusch, G. et al. Increased urinary excretion of kynurenic acid is associated with non-recovery from acute kidney injury in critically ill patients. BMC Nephrol 19, 44 (2018). doi.org/10.1186/s12882-018-0841-5). Additionally, plasma KYNA levels were consistently higher and plasma Trp levels were consistently lower in various AKI etiologies in mice (Wee, H, N., Liu, J. J., Ching, J., Kovalik, J, P., & Lin, S. C, (2021), The kynurenine pathway in acute kidney injury and chronic kidney disease. American Journal of Nephrology, 52(10-11), 771-787). These findings suggest that the presence of AKI may have an influence on the regulation of certain metabolic pathways. For instance, intrarenal and systemic inflammation have been routinely associated with AKI in previous studies (Rabb, H., Griffin, M. D., McKay, D. B., Swaminathan, S., Pickkers, P., Rosner, M. H., & Ronco, C. (2016). Inflammation in AKI: current understanding, key questions, and knowledge gaps. Journal of the American Society of Nephrology, 27(2), 371-379). This is consistent with our observation of higher urinary KYNA in preterm neonates with AKI compared to those without AKI, as IDO expression is upregulated in periods of inflammation and triggers the KP pathway (Schwarcz, R., & Stone, T. W. (2017). The kynurenine pathway and the brain: Challenges, controversies and promises. Neuropharmacology, 112, 237-247; Stone, T. W. (1993). Neuropharmacology of quinolinic and kynurenic acids. Pharmacological reviews, 45(3), 309-379; Turski, M. P., Turska, M., Paluszkiewicz, P., Parada-Turska, J., & Oxenkrug, G. F. (2013). Kynurenic acid in the digestive system—new facts, new challenges. International Journal of Tryptophan Research, 6, IJTR-S12536; Thdate, L, van Baren, N., Pilotte, L., Moulin, P., Larrieu, P., Renauld, J C., & Van den Eynde, B. J. (2015). Extensive profiling of the expression of the indoleamine 2, 3-dioxygenase 1 protein in normal and tumoral human tissues. Cancer immunology research, 3(2), 161-172; Mbongue, J. C., Nicholas, D. A., Torrez, T. W., Kim, N. S., Firek, A. F., & Langridge, W. H. (2015). The Role of Indoleamine 2, 3-Dioxygenase in Immune Suppression and Autoimmunity. Vaccines, 3(3), 703-729).

Conversely, Trp betaine has been shown to reduce the presence of certain inflammatory cytokines and decrease inflammation in endothelial cells (Sun, H., Zhu, X., Lin, W., Zhou, Y., Cai, W., & Qiu, L. (2017). Interactions of TLR4 and PPARγ, dependent on AMPK signalling pathway contribute to anti-inflammatory effects of vaccariae hypaphorine in endothelial cells. Cellular Physiology and Biochemistry, 42(3), 1227-1239). Thus, low Trp betaine levels may lead to increased intrarenal and systemic inflammation that is associated with AKI, and therefore augmentation of Trp betaine and suppression of IDO should be considered in future clinical trials involving preterm neonatal AKI.

Our urinary proteomic data, which showed no significant difference in preterm neonates with and without AKI is not consistent with existing studies. Elevations of urinary NGAL, Cystatin C, and B2M in preterm neonates with AKI have all been reported in literature (Hanna, NL, Brophy, P. D., Giannone, P. J., Joshi, M. S., Bauer, J. A., & RamachandraRao, S. (2016). Early urinary biomarkers of acute kidney injury in preterm infants. Pediatric research, 80(2), 218-223; Naunova-Timovska, S., Cekovska, S., Sahpazova, E., & Tasić, V. (2020). NEUTROPHIL GELATINASE-ASSOCIATED LIPOCALIN AS AN EARLY BIOMARKER OF ACUTE KIDNEY INJURY IN NEWBORNS. Acta clinica Croatica, 59(1), 55-62. doi.org/10.20471/acc.2020.59.01.07; Panza, R., Schirinzi, A., Baldassarre, M. E., Caravita, R., Laterza, R., Mascolo, E., & Laforgia, N. (2025). Evaluation of uNGAL and TIMP-2*IGFBP7 as early biomarkers of Acute Kidney Injury in Caucasian term and preterm neonates: a prospective observational cohort study. Italian Journal of Pediatrics, 51(1), 64). For example, some studies have reported median urinary NGAL levels of <1 ng/mL in preterm neonates without AKI and 4.24 ng/mL in preterm neonates with AKI. However, the Siemens BNII Nephelometer used in this study to detect urinary NGAL is for research use only (RUO), so the minimum detectable concentration of urinary NGAL was 9.38 ng/mL. Moreover, due to low sample volumes, a 5× dilution was performed. This increased the minimum detectable concentration of urinary NGAL to 46.9 ng/mL, so urinary NGAL values below 46.9 ng/mL may have been misinterpreted or not reported altogether. While our detection ranges of urinary CysC and urinary B2M were well within ranges previously reported in preterm neonates (Nakhjavan-Shahraki, B., Yousefifard, M., Ataei, N., Baikpour, M., Ataei, F., Bazargani, B., . . . & Hosseini, M. (2017). Accuracy of cystatin C in prediction of acute kidney injury in children; serum or urine levels: which one works better?A systematic review and meta-analysis. BMC nephrology, 18, 1-13; Abdullah, Kadam, P., Yachha, M., Srivastava, G., Pillai, A., & Pandita, A. (2022), Urinary beta-2 mnicroglobulin as an early predictive biomarker of acute kidney injury in neonates with perinatal asphyxia. European Journal of Pediatrics, 181(1), 281-286), these proteins are much more prone to degradation from proteases and temperature or pH fluctuations than is NGAL. Additionally, an increase in reactive oxygen species in urinary samples from AKI participants may also accelerate degradation of uCysC and uB2M (Himmelfarb, J., McMonagle, E., Freedman, S., Klenzak, J., McMenamin, E., Le, P., . . . & Picard Group. (2004). Oxidative stress is increased in critically ill patients with acute renal failure. Journal of the American Society of Nephrology, 15(9), 2449-2456).

The observed positive correlations between kidney hypoxia and urinary KYNA and CysC levels, as well as the negative correlation with urinary Trp betaine levels, are consistent with previously reported associations between RrSO₂ and AKI status. For instance, median RrSO₂ values have been reported to be lower on days two through seven in preterm neonates with AKI compared to those without AKI (Harer, M. W., Adegboro, C. O., Richard, L. J., & McAdams, R. M. (2021). Non-invasive continuous renal tissue oxygenation monitoring to identify preterm neonates at risk for acute kidney injury, Pediatric Nephrology, 36, 1617-1625). Given that study found elevations of urinary KYNA and CysC in preterm neonates with AKI, a positive correlation between kidney hypoxia and these biomarkers is logical. Similarly, decreased levels of urinary Trp betaine were found in preterm neonates with AKI, so a negative correlation between kidney hypoxia and urinary Trp betaine is consistent with these findings. Further studies are needed to more closely evaluate the relationship with kidney oxygenation measured by NIRS and urinary AKI biomarkers.

This study suggests there are elevated levels of urinary KYNA and decreased levels of urinary Trp betaine in preterm neonates with AKI, and that these biomarkers may be more sensitive in detecting AKI than are NGAL, CysC, and B2M proteins.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of detecting acute kidney injury metabolomic biomarkers (AKI) in a subject, comprising providing a urine sample from the subject, and detecting an AKI metabolomic biomarker in the urine sample.

2. The method of claim 1, comprising detecting two, three, four or more AKI biomarkers.

3. The method of claim 1, wherein the AKI metabolomic biomarker comprises two or more of furosemide, acesulfame, terephthalic acid, DL-stachydrine, kynurenic acid, hexaethylene glycol, 2′3′-cyclic GMP, panthenol/pantothenol, fluconazole, Val-Phe, tryptophan betaine, 4-hydroxyphenylacetic acid, Cyclo (Pro_Leu/ile), tricarballylic acid, tyramine, decanoylcarnitine, norfentanyl, 3-indoxyl sulphate, cyclo (pro_Val), methylxanthine, DL-carnitine, 1,2,3-propanetricarboxylic acid, cyclo (Pro_Tyr), acetominophen, DL-dopa, and combinations thereof.

4. The method of claim 1, wherein the AKI metabolomic biomarker comprises at least one elevated biomarker selected from furosemide, acesulfame, terephthalic acid, DL-stachydrine, kynurenic acid, hexaethylene glycol, 2′3′-cyclic GMP, panthenol/pantothenol, fluconazole, and Val-Phe; and at least one decreased biomarker selected from tryptophan betaine, 4-hydroxyphenylacetic acid, cyclo (Pro_Leu/ile), tricarballylic acid, tyramine, decanoylcarnitine, norfentanyl, 3-indoxyl sulphate, cyclo (pro_Val), methylxanthine, DL-carnitine, 1,2,3-propanetricarboxylic acid, cyclo (Pro_Tyr), acetominophen, and DL-dopa.

5. The method of claim 1, wherein the at least one elevated biomarker comprises kynurenic acid and the at least one decreased biomarker comprises tryptophan betaine.

6. The method of claim 1, wherein the subject is a neonate or an infant born prematurely.

7. The method of claim 1, wherein the subject is a child or an adult who was born prematurely.

8. The method of claim 1, wherein the subject is an infant, a child or an adult suspected of having AKI.

9. The method of claim 1, wherein a level of the AKI metabolomic biomarker is increased or decreased by 10%, 20%, 30%, 40%, 50%, 60%, 70%, 80%, 90% or more relative to a level of the AKI metabolomic biomarker in a normal control.

10. The method of claim 1, wherein the AKI metabolomic biomarker is furosemide, acesulfame, hexaethylene glycol, kynurenic acid, kynurenine, acetaminophen glucuronide, ceftazidime, fluconazole, ampicillin, DL-carnitine, tyramine, decanoylcarnitine, bilirubin, propionylcarnitine, cyclo(Pro_Tyr), tryptophan betaine, cyclo(Pro_Leu/Ile), cyclo(Pro_Val), 1, 2, 3, -propanetricarboxylic acid, 3-indoxyl sulfate, norfentanyl, panthenol/pantothenol, Val-Phe, 2′, 3′-cyclic GMP/3′,5′-cyclic GMP, DL-cystine, methylxanthine, DL-DOPA, pantothenic acid, acetylcholine, S-adenosylmethionine, succinic acid, dehydroascorbic acid, homovanillic acid, DL-glutamic acid, L-tyrosine, citric acid, pyroglutamic acid, pyruvic acid, L-tryptophan, choline, methylmalonic acid, L-valine, phenylacetylglutamine, ornithine, creatine, 3-ureidopropionic acid, panthenoic acid, carnosine, urocanic acid, N-acetylneuraminic acid, xanthosine, inosine, hypoxanthine, uric acid, xanthine, betaine, uridine, orotic acid, nicotinamide, N-Acetyl-L-aspartic acid, phenylalanine, riboflavin, thiamine, glycocholic acid, 5′-methylthioadenosine, 1,3,7-trimethyluric acid, theobromine, caffeine, paraxanthine, theophylline, 4-hydroxyphenylacetic acid, 2-furoylglycine, Asp_Glu, 2-aminoadipic acid, proline, sebacic acid, 2-furoic acid, 4-acetamidobutanoic acid, mandelic acid, 3,5-dimethoxybenzoic acid, N-acetylputrescine, N-acetyl-L-tyrosine, methylsuccinic acid, azelaic acid, labetalol, terephthalic acid, DL-stachydrine, metoclopramide, 2-aminoadipic acid, 4-acetamidobutanoic acid, hippuric acid, phthalic acid, Ala-Pro/Pro-Ala, hydroxyoctanoic acid, or a combination thereof.

11. The method of claim 1, wherein detecting the AKI metabolomic biomarker comprises mass spectrometry, chromatography, spectroscopy, a chemical method, an immunoassay, or a combination thereof.

12. The method of claim 11, wherein detecting uses a reagent for detecting, binding, or capturing the AKI metabolomic biomarker.

13. The method of claim 1, further comprising detecting neutrophil gelatinase-associated lipocalin in the urine sample.

14. The method of claim 1, further comprising near-infrared spectroscopy (NIRS) monitoring to detect kidney hypoxia.

15. The method of claim 1, further comprising administering an AKI therapy to the subject.

16. The method of claim 15, wherein the AKI therapy is peritoneal dialysis, continuous kidney support therapy, theophylline, caffeine, a diuretic, avoidance of nephrotoxic medications, or a combination thereof.

17. The method of claim 1, wherein, prior to providing the urine sample from the subject, the method comprises administering an AKI therapy to the subject, wherein detecting the AKI metabolomic biomarker in the urine sample is done after administering an AKI therapy to the subject.

18. An article comprising a reagent for detection of an AKI metabolomic biomarker in a urine sample, wherein the AKI metabolomic biomarker is furosemide, acesulfame, hexaethylene glycol, kynurenic acid, kynurenine, acetaminophen glucuronide, ceftazidime, fluconazole, ampicillin, DL-carnitine, tyramine, decanoylcarnitine, bilirubin, propionylcarnitine, cyclo(Pro_Tyr), tryptophan betaine, cyclo(Pro_Leu/Ile), cyclo(Pro_Val), 1, 2, 3, -propanetricarboxylic acid, 3-indoxyl sulfate, norfentanyl, panthenol/pantothenol, Val-Phe, 2′, 3′-cyclic GMP/3′,5′-cyclic GMP, DL-cystine, methylxanthine, DL-DOPA, pantothenic acid, acetylcholine, S-adenosylmethionine, succinic acid, dehydroascorbic acid, homovanillic acid, DL-glutamic acid, L-tyrosine, citric acid, pyroglutamic acid, pyruvic acid, L-tryptophan, choline, methylmalonic acid, L-valine, phenylacetylglutamine, ornithine, creatine, 3-ureidopropionic acid, panthenoic acid, carnosine, urocanic acid, N-acetylneuraminic acid, xanthosine, inosine, hypoxanthine, uric acid, xanthine, betaine, uridine, orotic acid, nicotinamide, N-Acetyl-L-aspartic acid, phenylalanine, riboflavin, thiamine, glycocholic acid, 5′-methylthioadenosine, 1,3,7-trimethyluric acid, theobromine, caffeine, paraxanthine, theophylline, 4-hydroxyphenylacetic acid, 2-furoylglycine, Asp_Glu, 2-aminoadipic acid, proline, sebacic acid, 2-furoic acid, 4-acetamidobutanoic acid, mandelic acid, 3,5-dimethoxybenzoic acid, N-acetylputrescine, N-acetyl-L-tyrosine, methylsuccinic acid, azelaic acid, labetalol, terephthalic acid, DL-stachydrine, metoclopramide, 2-aminoadipic acid, 4-acetamidobutanoic acid, hippuric acid, phthalic acid, Ala-Pro/Pro-Ala, hydroxyoctanoic acid, or a combination thereof.

19. The method of claim 18, wherein the article is a diaper, a foley catheter, a lateral flow test, or an absorbent cotton pad.

20. A method of identifying metabolomic biomarkers associated with acute kidney injury (AKI) in preterm neonates, comprising
collecting urine samples from a population of preterm neonates, the population comprising a first subpopulation having no evidence of AKI, and a second subpopulation having AKI;
performing a metabolomic analysis on the urine samples;
comparing the first population and the second population and identifying metabolites significantly higher or significantly lower in the second subpopulation compared to the first subpopulation; and
validating the metabolites to provide the metabolomic biomarkers.
